(19) United States
(12) Patent Application Publication
(10) Pub. No.: US 2024/0034979 A1
(43) Pub. Date: Feb. 1, 2024

(54) BIOREACTOR APPARATUS AND SYSTEM

(71) Applicant: NATIONAL CENTRAL UNIVERSITY, Taoyuan City (TW)

(72) Inventors: Ching-Yun CHEN, Taoyuan City (TW); Feng-Huei LIN, Taoyuan City (TW); Jui-Sheng SUN, Taoyuan City (TW); Cherng-Jyh KE, Taoyuan City (TW); Fu-Chiang YEH, Taoyuan City (TW); Chun-Yi PENG, Taoyuan City (TW); Jia-Ci JHANG, Taoyuan City (TW); Kai-Wei LIN, Taoyuan City (TW); Qi-Hong HONG, Taoyuan City (TW); Yu-Syuan DING, Taoyuan City (TW)

(73) Assignee: NATIONAL CENTRAL UNIVERSITY, Taoyuan City (TW)

(21) Appl. No.: 18/359,305

(22) Filed: Jul. 26, 2023

(30) Foreign Application Priority Data
Jul. 28, 2022 (TW) 111128412

(51) Int. Cl. C12M 1/00

(52) U.S. Cl. CPC C12M 29/18; C12M 29/02; C12M 23/58

(57) ABSTRACT

Provided is a bioreactor apparatus including: a liquid storage chamber for storing a culture medium; a pump for providing pressure to drive the flow of the culture medium; a plurality of culture chambers providing an accommodating space to accommodate the culture medium and a cell to be cultured; and a pipeline connecting the liquid storage chamber, the pump, and the culture chamber to form a closed loop. Also provided is a bioreactor system including the bioreactor apparatus of the present disclosure, the culture medium, and a cell. Further provided is a method for culturing a cell or an organoid by the bioreactor apparatus of the present disclosure. The bioreactor apparatus, bioreactor system, and method of the present disclosure can form a biomimetic circulatory system, which is beneficial for simulating and evaluating the complex microenvironment and physiological mechanism in the living body.

### CROSS-REFERENCE TO RELATED APPLICATIONS

This application claims priority to Taiwan Application No. 111128412, filed on Jul. 28, 2022. The entirety of the above-mentioned patent application is hereby incorporated by reference herein and made a part of this specification.

### BACKGROUND

### 1. Technical Field

The present disclosure relates to a bioreactor apparatus and system, and particularly to an ex vivo testing platform that can simultaneously culture different cells and simulate the biological circulatory system.

### 2. Description of Relevant Art

Recently, there is a growing trend of reduction in animal experiments, with a societal expectation to replace the animal experiments with in vitro testing methods. Microfluidic biochip is one of the earlier developed in vitro testing methods, which utilizes methods such as photolithography etching to form patterns on a chip. Such patterns can be used as culture areas and channels. Different cells are cultured in different culture areas that are connected to each other via the channels, thereby constructing a biomimetic model simulating in vivo environment. One of the characteristics of the microfluidic biochips is their small volume, such that the amount of samples required is small.

However, limited by the small volume, the microfluidic biochip is usually for two-dimensional culture when culturing cells, as the space thereof is insufficient for three-dimensional culture. Therefore, the cultural space of the microfluidic biochip is too limited to scale up the number of cultured cells or the volume of organoids, leading to more difficulties in forming a system that simulates the actual inner workings of a living body. Furthermore, since the patterns on the chip are prefabricated by precision instrument, the culture areas and channels thereon cannot be increased, decreased, or adjusted afterward at the time of application according to actual needs, and the additional substances also cannot be added to the specific culture area. Hence, the microfluidic biochip has less flexibility in application.

A bioreactor is an apparatus that can culture organisms such as human cells, animal cells, plant cells, or microorganisms. The organisms undergo biochemical reactions in the bioreactor that simulates biological functions. For example, the yeasts are cultured in the bioreactor to undergo a fermentation reaction and to convert glucose into ethanol; Another example is culturing target cells in a bioreactor to proliferate and obtain a sufficient large amount of the target cells. However, the purpose of the bioreactors known in the art is still the obtainment of a large amount of target substances or cells, without designs for simulating the biological circulatory system, let alone evaluation of the complex microenvironment and physiological mechanism in the living body.

Accordingly, there is an urgent and unmet need in the art to provide a biomimetic apparatus and system that can solve the problems mentioned above.

### SUMMARY

To solve the aforementioned problems, the present disclosure provides a bioreactor apparatus, comprising: a liquid storage chamber for storing a culture medium; a pump; a culture chamber having an accommodating space for accommodating the culture medium and cells to be cultured; and a pipeline connected to the liquid storage chamber, the pump, and the culture chamber to form a closed loop, wherein the pump is configured to provide pressure for driving the flow of the culture medium within the closed loop.

In at least one embodiment of the present disclosure, the bioreactor apparatus comprises a plurality of the culture chambers connected to each other in series, in parallel, or a combination thereof.

In at least one embodiment of the present disclosure, each of the culture chambers has an opposite top part and bottom part. The top part has an inlet allowing the culture medium to enter the accommodating space of the culture chambers, and the top part also has an outlet allowing the culture medium at the bottom part to leave the accommodating space of the culture chambers.

In at least one embodiment of the present disclosure, the culture chamber comprises a lid and a tube, and the lid is disposed on the top part and forms the inlet and the outlet.

In at least one embodiment of the present disclosure, the inlet is disposed obliquely relative to the lid, and the outlet is perpendicular to the lid. In some embodiments, an angle of 30 degrees to 60 degrees is formed between the lid and the inlet.

In at least one embodiment of the present disclosure, the culture chamber further includes an inner pipe having an opposite first opening and a second opening, wherein the first opening is connected to the outlet, and the second opening is located at the bottom part of the culture chamber.

In at least one embodiment of the present disclosure, the culture medium passes through the inlet and drips into the accommodating space in the culture chamber. The culture medium in the accommodating space enters the inner pipe through the second opening of the inner pipe, and leaves the accommodating space through the first opening of the inner pipe and the outlet.

In at least one embodiment of the present disclosure, the culture chamber further comprises an outer pipe having an opposite third opening and fourth opening, wherein the third opening is connected to the inlet.

In at least one embodiment of the present disclosure, the diameter of the outer pipe is larger than the diameter of the inner pipe, and the outer pipe is sleeved around the inner pipe.

In at least one embodiment of the present disclosure, the diameter of the third opening is larger than the diameter of the fourth opening.

In at least one embodiment of the present disclosure, the outer pipe has a hole positioned on the wall of the outer pipe. In some embodiments, the outer pipe has a plurality of holes positioned on a wall of the outer pipe to allow the culture medium to flow out of the outer pipe through the holes.

In at least one embodiment of the present disclosure, the holes are arranged at intervals of 30 to 180 degrees in a radial direction of the outer pipe. In some embodiments, the holes are asymmetrically arranged.

In at least one embodiment of the present disclosure, the culture medium passes through the inlet and the third opening of the outer pipe into the outer pipe, flowing to the outside of the outer pipe through the holes. The culture medium in the accommodating space enters the inner pipe through the second opening of the inner pipe, and leaves the accommodating space through the first opening of the inner pipe and the outlet.

In at least one embodiment of the present disclosure, any two of the liquid storage chamber, the pump, and the culture chamber are connected to each other by the pipeline.

In at least one embodiment of the present disclosure, the bioreactor apparatus further comprises a connector for connecting the pipelines, and the connector has an opening openable and closable for adding additional substances into the pipelines.

In at least one embodiment of the present disclosure, the pump is configured to provide a constant pressure, a periodic pressure, or a pulsatory pressure.

In at least one embodiment of the present disclosure, the bioreactor apparatus serves as an ex vivo testing platform.

The present disclosure further provides a bioreactor system that is a closed-loop system, comprising: the bioreactor apparatus of the present disclosure; a culture medium stored in the liquid storage chamber of the bioreactor apparatus to be transported to each of the culture chambers through the pipelines; and cells cultured in the culture chambers of the bioreactor apparatus.

In at least one embodiment of the present disclosure, the cells are cultured in suspension culture or adhesion culture. In certain embodiments, the cells cultivated in each of the culture chambers are different from one another.

In at least one embodiment of the present disclosure, the culture chamber includes a liquid section and a gas section. The liquid section comprises the culture medium for culturing cells.

In at least one embodiment of the present disclosure, the liquid section further includes a scaffold.

In at least one embodiment of the present disclosure, the scaffold is at least one selected from the group consisting of three-dimensional porous calcium alginate crosslinked bioscaffolds, three-dimensional porous collagen bioscaffolds, three-dimensional porous gelatin bioscaffolds, three-dimensional magnetic porous bioscaffolds, three-dimensional alginate/gelatin combined cell carriers, three-dimensional magnetic cell carriers.

In at least one embodiment of the present disclosure, the bioreactor system further includes a sensor for sensing the ingredients of the culture medium.

In at least one embodiment of the present disclosure, the ingredients of the culture medium to be sensed are at least one selected from the group consisting of proteins, exosomes, glucose, hydrogen ion, oxygen, and nitrogenous wastes. In at least one embodiment of the present disclosure, the proteins include growth factors, paracrine factors, antibodies, or other cell-derived water soluble proteins.

In at least one embodiment of the present disclosure, the culture chamber has a large enough accommodating space, so that the volume of the culture medium and the organoids can be controlled by adjusting the ratio of the liquid section and the gas section, and a two-dimensional or a three-dimensional culture can be readily realized.

In at least one embodiment of the present disclosure, the outlet and inlet of the culture chamber and the outer pipe are additionally designed to control the liquid surface disturbances caused by the input culture medium in the liquid section of the culture chamber, so as to further explore the relationship between the disturbances and the cells/organoids.

In at least one embodiment of the present disclosure, each of the components of the bioreactor is detachable. Therefore, the components can be replaced or changed at any time according to the need during the application, which is very convenient.

In at least one embodiment of the present disclosure, additional substances can be added into the pipelines by a three-way pipe disposed thereon to modify the culture chamber's microenvironment and to observe and analyze the result, to realize applications such as drug screening, environment testing and food safety testing. The exemplary additional substance may be an additional culture medium, drugs, toxicants, samples, cytokines, or growth factors.

The present disclosure also provides a method for culturing a cell or an organoid, comprising providing the bioreactor apparatus of the present disclosure; and culturing the cell or the organoid in the culture chamber.

In at least one embodiment of the present disclosure, the method further comprises loading the culture medium in the liquid storage chamber and the culture chamber; and starting the pump to provide pressure to drive the flow of the culture medium in the closed loop. In some embodiments, the pressure is a constant pressure, a periodic pressure, or a pulsatory pressure.

In conclusion, the bioreactor apparatus and system of the present disclosure has a plurality of culture chambers that can simultaneously culture the different cells. Moreover, the preset disclosure can construct a suitable environment for culturing different cells or organoids with good viability by disposing of the scaffolds, controlling the concentration of various ingredients or pH value of the culture medium, adjusting the temperature or the flow rate of the culture medium in the pipelines, etc. Furthermore, the culture chambers, the liquid storage chamber, and the pump in the bioreactor apparatus and system of the present disclosure are interconnected by the pipelines to simulate the signal transduction and interaction between various cells, tissues, or organs within a living body, thereby realizing an ex vivo biomimetic model.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic diagram illustrating the bioreactor apparatus and system according to an embodiment of the present disclosure.

FIG. 2 is a perspective view illustrating the culture chamber according to an embodiment of the present disclosure.

FIG. 3 is an exploded drawing illustrating the culture chamber according to an embodiment of the present disclosure.

FIG. 4 is a cross-sectional view illustrating the culture chamber according to an embodiment of the present disclosure.

FIG. 5A and FIG. 5B are side views illustrating the outer pipe according to an embodiment of the present disclosure.

FIG. 5C and FIG. 5D are perspective views illustrating the outer pipe according to another embodiment of the present disclosure.

FIG. 6 is a schematic diagram illustrating the liquid flow in the culture chamber during operation according to an embodiment of the present disclosure.

FIG. 7 is a schematic diagram illustrating the bioreactor apparatus and system according to another embodiment of the present disclosure.

FIG. 8A to FIG. 8C are actual images of the bioreactor apparatus according to an embodiment of the present disclosure.

FIG. 9 is an actual image of the bioreactor apparatus according to an embodiment of the present disclosure.

FIG. 10 is an actual image of the culture chamber according to an embodiment of the present disclosure.

FIG. 11A to FIG. 11C are cell microscopy images of cells cultured in the culture chamber of the bioreactor apparatus according to an embodiment of the present disclosure. In FIG. 11A, green fluorescence shows F-actins labeled by Phalloidin, and blue fluorescence shows cell nuclei stained by Hoechst 33342. In FIG. 11B, green fluorescence shows F-actins labeled by Phalloidin; red fluorescence shows healthy mesenchymal stem cells labeled by 5,5,6,6-Tetrachloro-1,1,3,3-tetraethylbenzimidazolylcarbocyanine iodide (a JC-1 mitochondria dye); blue fluorescence shows cell nuclei stained by Hoechst 33342. In FIG. 11C, green fluorescence is live cells labeled by Calcein AM, and red fluorescence shows dead cells labeled by Propidium Iodide (PI).

FIG. 12 is cell microscopy images of cells cultured in the culture chamber of the bioreactor apparatus according to an embodiment of the present disclosure.

### DETAILED DESCRIPTION

The following descriptions of the embodiments illustrate implementations of the present disclosure, and those skilled in the art of the present disclosure can readily understand the advantages and effects of the present disclosure in accordance with the contents herein. However, the embodiments of the present disclosure are not intended to limit the scope of the present disclosure. The present disclosure can be practiced or applied by other alternative embodiments, and every detail included in the present disclosure can be changed or modified in accordance with different aspects and applications without departing from the essentiality of the present disclosure.

The features such as a ratio, structure, and dimension shown in drawings accompanied with the present disclosure are simply used to cooperate with the contents disclosed herein for those skilled in the art to read and understand the present disclosure, rather than to limit the scope of implementation of the present disclosure. Thus, in the case that does not affect the purpose of the present disclosure and the effect brought by the present disclosure, any change in proportional relationships, structural modification, or dimensional adjustment should fall within the scope of the technical contents disclosed herein.

As used herein, “comprising” (and any variant or conjugation thereof, such as “comprise” or “comprises”), “including” (and any variant or conjugation thereof, such as “include” or “includes”), or “having” (and any variant or conjugation thereof, such as “have” or “has”) a specific element, unless otherwise specified, may include other elements such as components, ingredients, structures, regions, portions, devices, systems, steps, or connection relationships rather than exclude those elements.

The terms “on,” “upper,” “under,” “lower,” “front,” and “rear” described herein are simply used to clarify the embodiments of the present disclosure, rather than used to limit the scope of implementation of the present disclosure. Adjustments, interchanges, and alteration of relative positions and relationships thereof should be considered within the scope of implementation of the present disclosure if the technical contents of the present disclosure are not substantially changed.

The terms “first,” “second,” “third,” “fourth,” etc., used herein are simply used to describe or distinguish elements such as components, ingredients, structures, regions, portions, devices, or systems, rather than used to limit the scope of implementation of the present disclosure or to limit the spatial order of the elements. In addition, unless otherwise specified, the singular forms “a” and “the” used herein also include plural forms, and the terms “or” and “and/or” used herein are interchangeable.

The numeral ranges used herein are inclusive and combinable, and any numeral value that falls within the numeral scope herein can be taken as a maximum or minimum value to derive the sub-ranges therefrom. For example, it should be understood that the numeral range from “30 degrees to 60 degrees” comprises any sub-ranges between the minimum value of 30 degrees and the maximum value of 60 degrees, such as the sub-ranges from 40 degrees to 60 degrees, from 30 degrees to 50 degrees, and from 35 degrees to 55 degrees. Furthermore, any multiple numeral points used herein can be chosen as a maximum or minimum value to derive the numeral ranges therefrom. For example, 0.1 mm, 5 mm, and 10 mm can derive the numeral ranges of 0.1 to 5 mm, 0.1 to 10 mm, or 5 to 10 mm.

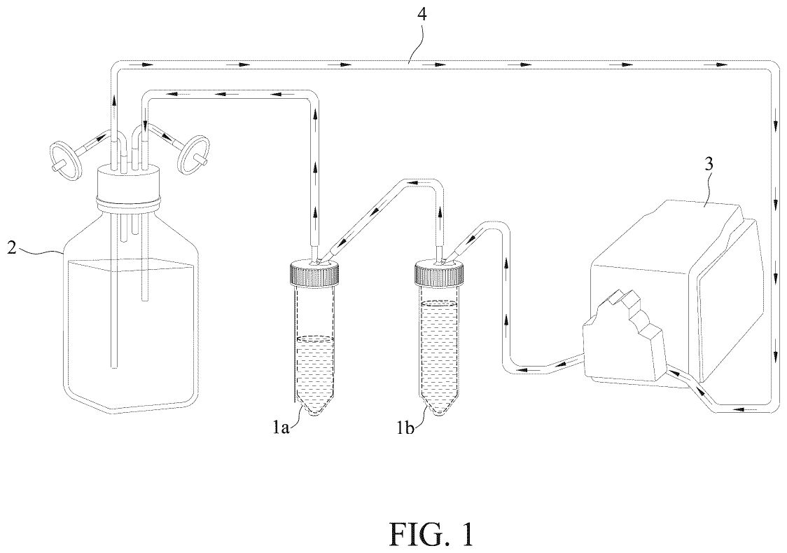

FIG. 1 shows the bioreactor apparatus according to at least one embodiment of the present disclosure, including a liquid storage chamber 2, a culture chamber 1a, a culture chamber 1b, pipelines 4, and a pump 3. It should be noted that the quantity and connection of each of the parts are exemplary and can be increased, decreased, or altered according to actual needs. As shown in FIG. 1, the liquid storage chamber 2 is used for storing a culture medium; the pipelines 4 connect the liquid storage chamber 2, the pump 3, the culture chamber 1a, and the culture chamber 1b, so that the culture medium can be transported along the pipelines 4; the pump 3 provides a power source in the form of pressure to drive the transportation of the culture medium. In the embodiment, the culture medium leaves the liquid storage chamber 2 and enters the pipelines 4 to be transported to the culture chambers 1a and 1b, and then flows from the culture chambers 1a and 1b back to the liquid storage chamber 2, forming a closed-loop system.

The term “connect” or a conjugation thereof used herein refers to a plurality of elements directly joined or indirectly joined together. The term “directly joined” means that a plurality of elements are joined together by direct contact with each other, and the term “indirectly joined” means that a plurality of elements are joined together by at least one connecting component. The meaning of “connecting” used herein comprises compact joining, bonding, embedding, screwing, fastening, clamping, attaching, puncturing, clipping, disposing, integrated molding, or a combination of two or more thereof. Furthermore, the term “connector” used herein refers to the element that can achieve the aforementioned means of “connecting.”

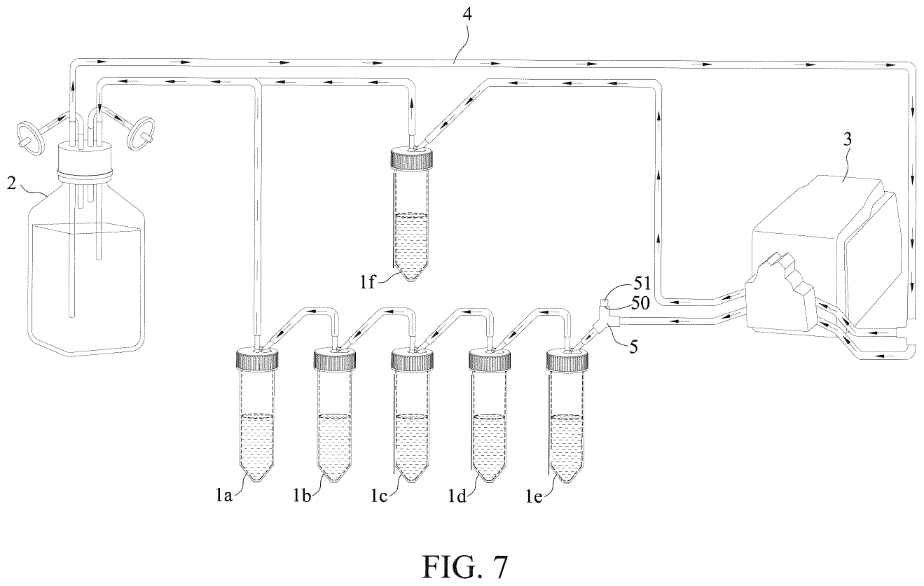

In at least one embodiment of the present disclosure, the plurality of the culture chambers are interconnected with each other by the pipelines, and thus the culture medium therein can serve as a medium for signal transduction. For example, the culture medium in the culture chamber 1b can carry substances secreted by cells cultured therein and flow to the culture chamber 1a, thereby making the substances act on/influence cells cultured in the culture chamber 1a. By the aforementioned mechanism, the bioreactor apparatus of the present disclosure can simulate signal transduction in a living body. Although FIG. 1 only shows two culture chambers 1a and 1b, the number of the culture chambers can be more in other embodiments. For instance, FIG. 7 shows a bioreactor apparatus having six culture chambers 1a, 1b, 1c, 1d, 1e, and if to simulate more complex and detailed signal transduction and interaction in a living body. Also, these culture chambers can be connected in series, parallel, or a combination thereof, so that the bioreactor apparatus is more analogous to the real condition in the living body. FIG. 7 exemplarily shows a setup having a group of five culture chambers (1a, 1b, 1c, 1d, and 1e) connected in series and another culture chamber if connected to the group in parallel, but the serial or parallel configuration is not limited thereto.

The number of the liquid storage chamber 2 may be one or more. For example, the first liquid storage chamber may be the liquid storage chamber 2 in FIG. 1 and FIG. 7, and the liquid storage chamber other than the first liquid storage chamber can serve as a spare liquid storage chamber or a dilution bottle. The number of the pump 3 may also be one or more, and the pump 3 may be a multi-channel pump as shown in FIG. 7 for providing access to a plurality of channels. The pump 3 can provide pressures to different pipelines 4, respectively, to realize stable transportation. Furthermore, the pump can also provide a constant pressure, a periodic pressure, a pulsatory pressure, etc. Various types of pressure can be adapted to different cells and different experimental or cultural needs.

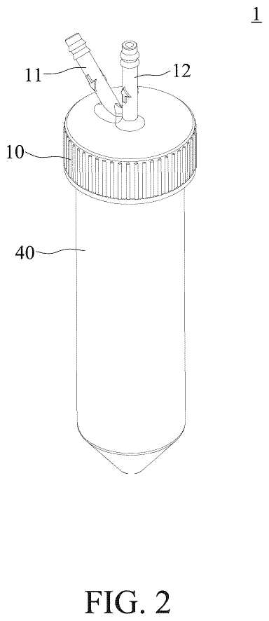

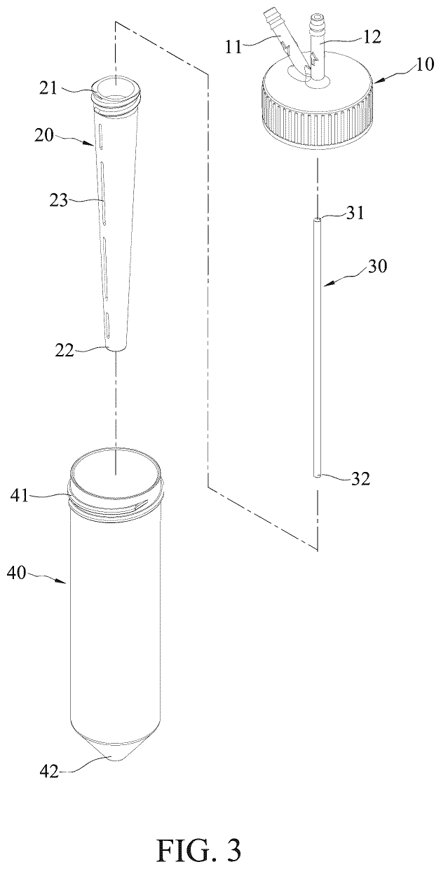

The bioreactor apparatus of the present disclosure can serve as an ex vivo testing platform. FIG. 2 to FIG. 4 show the culture chamber 1 of the bioreactor apparatus according to an embodiment of the present disclosure. The culture chamber 1 has a lid 10 and a tube 40. The lid 10 has an inlet 11 and an outlet 12. The tube 40 has a top part 41 and a bottom part 42, the top part 41 is an opened part for connecting with the lid 10, and the bottom part 42 is a sealed part. Thus, the tube 40 can provide an accommodating space to accommodate the culture medium and the cells. The tube 40 can be connected to the lid 10, e.g., by a first joint portion 15 formed on the lid 10. Also, the top part 41 of the tube 40 and the first joint portion 15 have external threads and internal threads, respectively, and the two are combined with each other by screwing. However, the lid 10 may be connected to the tube 40 by other means in other embodiments. Referring again to FIG. 1 to FIG. 3, the inlet 11 and the outlet 12 of the lid 10 are used for connecting the pipelines 4. As indicated by an arrow on the inlet 11 in FIG. 2 and FIG. 3, the culture medium outside the culture chamber 1 passes through the pipelines 4 and enters the accommodating space of the culture chamber 1 via the inlet 11. Moreover, as indicated by an arrow on the outlet 12 in FIG. 2 and FIG. 3, the culture medium in the accommodating space leaves the culture chamber 1 through the outlet 12 and continues to be transported by the pipelines 4.

In at least one embodiment of the present disclosure, the disposal of the inlet 11 differs from that of the outlet 12. For example, as shown in FIG. 2, the inlet 11 is disposed obliquely relative to the lid 10, while the outlet 12 is disposed perpendicularly relative to the lid 10. In the aforementioned oblique disposal, the angle between the lid 10 and the inlet 11 may be 30 degrees to 60 degrees, 40 degrees to 60 degrees, or 45 degrees to 60 degrees, but the present disclosure is not limited thereto. Specifically, the angle between the lid 10 and the inlet 11 may be 30 degrees, 35 degrees, 40 degrees, 45 degrees, 50 degrees, 55 degrees, or 60 degrees. On the other hand, the aforementioned perpendicular disposal means that the angle between the lid 10 and the inlet 11 may be about 85 degrees to about 95 degrees, such as, but not limited to, 85 degrees, 86 degrees, 87 degrees, 88 degrees, 89 degrees, 90 degrees, 91 degrees, 92 degrees, 93 degrees, 94 degrees, or 95 degrees.

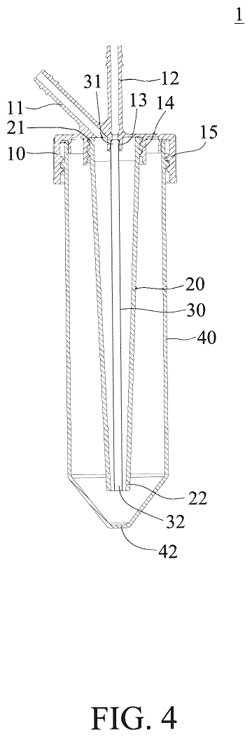

In at least one embodiment of the present disclosure, the culture chamber 1 further includes an inner pipe 30 that can be disposed in the accommodating space inside the culture chamber 1 as shown in FIGS. 3 and 4. The inner pipe 30 has an opposite first opening 31 and second opening 32. The first opening 31 can be connected to the outlet 12, and the second opening 32 can be disposed near the bottom part 42 of the tube 40 to transport the culture medium near the bottom part 42 to the outside through the inner pipe 30. The first opening 31 of the inner pipe 30 and the outlet 12 can be connected by, for example, a second joint part 13 with a protrusion formed on the lid 10, and the first opening 31 of the inner pipe 30 is inserted into the second connector 13. However, the inner pipe 30 can be connected to the outlet 12 by other means in other embodiments. In at least one embodiment of the present disclosure, the diameter of the inner pipe 30 may be from 0.1 mm to 10 mm, such as 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm. In some embodiments of the present disclosure, the material of the inner pipe 30 may be a biocompatible plastic polymer or stainless steel.

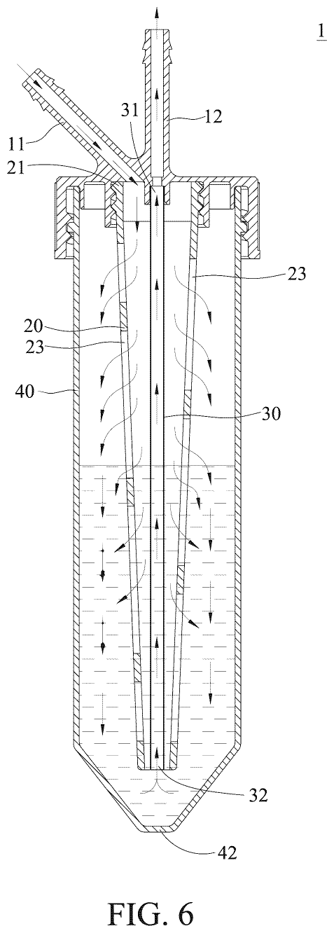

As shown in FIG. 6, in a culture chamber 1 including the inner pipe 30 according to at least one embodiment, an external culture medium passes through the inlet 11 and drips into the accommodating space in the culture chamber 1. The culture medium at the bottom part 42 of the accommodating space enters the inner pipe 30 through the second opening 32 of the inner pipe 30 and leaves the accommodating space through the first opening 31 of the inner pipe 30 and the outlet 12, and then is transported to the next culture chamber or the liquid storage chamber 2 through the pipelines 4.

As shown in FIGS. 3, 4, and 6, the culture chamber 1 can further include an outer pipe 20 that may be also disposed in the accommodating space inside the culture chamber 1. The outer pipe 20 has an opposite third opening 21 and a fourth opening 22. The third opening 21 can be connected to the inlet 11, so that the external culture medium can enter the outer pipe 20 through the inlet 11, and the fourth opening 22 can be disposed near the bottom part 42 of the tube 40. The third opening 21 and the inlet 11 can be connected by, for example, a third joint part 14 with a protrusion formed on the lid 10. Also, the third opening 21 and the third connector 14 have an external threads and an internal threads, respectively, and thus the two can be combined with each other by screwing. However, the outer pipe 20 may be connected to the inlet 11 by other means in other embodiments. For instance, the outer pipe 20 and the inlet 11 can be connected by means of the aforementioned method for connecting the inner pipe 30 and the outlet 12, but the present disclosure is not limited thereto.

In at least one embodiment of the present disclosure, the diameter of the outer pipe 20 is larger than the diameter of the inner pipe 30, and the outer pipe 20 is sleeved around the periphery of the inner pipe 30, as shown in FIGS. 3, 4, and 6. In some embodiments, the diameter of the third opening 21 of the outer pipe 20 may be larger than the diameter of the fourth opening 22. For example, the diameter of the outer pipe 20 gradually decreases along the axial direction from the third opening 21 to the fourth opening 22 to form a cone-like shape. Accordingly, when the external culture medium enters into the outer pipe 20 from the inlet 11, it can first contact the wall of the outer pipe 20 due to the cone-like shape, and then slide along the wall toward the liquid surface of the culture medium inside the culture chamber 1. As such, the vertical dripping of the external culture medium from the inlet 11 can be avoided, which eliminates disturbance of the liquid surface. In an embodiment, with the aforementioned oblique disposal of the inlet 11, when the external culture medium enters into the outer pipe 20 from the inlet 11, the external culture medium can better contact the wall of the outer pipe 20 and slide along the wall. In at least one embodiment of the present disclosure, the diameter of the third opening 21 and the diameter of the fourth opening 22 of the outer pipe 20 may be from 0.2 mm to 20 mm, such as 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, or 20 mm. In some embodiments, the material of the outer pipe 20 may be a biocompatible plastic polymer or stainless steel.

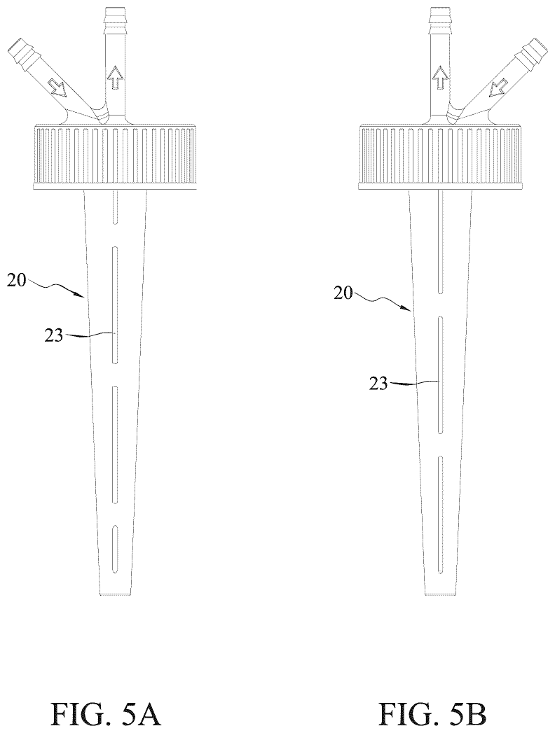

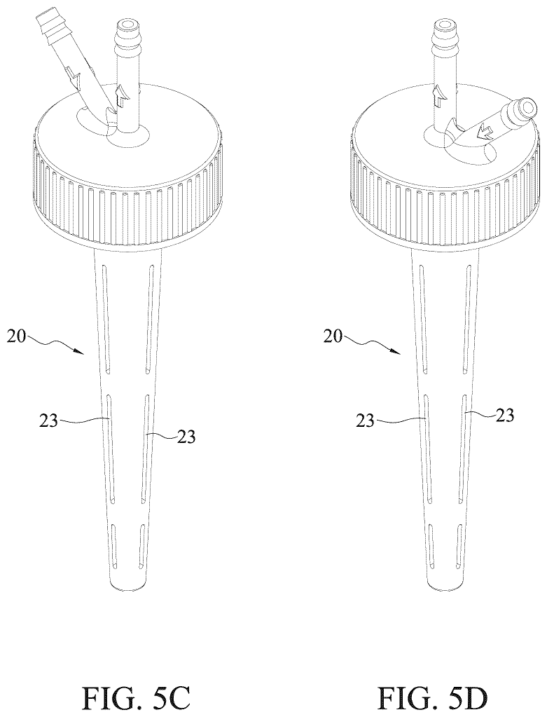

In at least one embodiment of the present disclosure, the wall of the outer pipe 20 has holes 23, as shown in FIGS. 5A to 5D and FIG. 6. When the external culture medium enters into the outer pipe 20 from the inlet 11, it can pass through the holes 23 and flow to the outside of the outer pipe 20 in the accommodating space of the culture chamber 1. In some embodiments, as the external culture medium flows in from the inlet 11 and slides along the wall of the outer pipe 20, the external culture medium can flow through the holes 23 and flow out of the outer pipe 20 via the holes 23, thereby avoiding the vertical dripping of the external culture medium from the inlet 11 to the liquid surface of the culture medium in the culture chamber 1 and eliminating the liquid surface disturbance.

The liquid surface disturbance of the culture medium is one of the factors that affects cell culture or differentiation. The present disclosure realizes the control of the liquid surface disturbance by the disposal of the outer pipe 20, hence can better analyze, control, and observe the condition of cell culture. In at least one embodiment, the outer pipe 20 may or may not be provided in the culture chamber according to actual needs, such as in response to the characteristics of different cells. In some embodiments, the outer pipe 20 is not disposed therein to let the external culture medium drip vertically from the inlet 11 to the liquid surface of the culture medium inside the culture chamber 1, thereby enhancing the liquid surface disturbance.

The number, shape, and arrangement of the holes 23 in the present disclosure are not limited and can be set according to the actual needs. In at least one embodiment, the holes 23 can be plural to increase the chance of the external culture medium flowing through the holes 23 on the wall of the outer pipe 20. In some embodiments, the holes 23 can be slit-shaped as shown in FIG. 3 and FIGS. 5A to 5D. In other embodiments, the shape of the holes 23 includes, but not limited to, polygonal, curved, irregular, or any combination of all or part of the above, such as triangle, rectangle, diamond, trapezoid, parallelogram, circle, oval, egg-shape, or any combination of all or part of the above. In certain embodiments, the holes 23 can be arranged at intervals of 30 degrees to 180 degrees in a radial direction of the outer pipe 20, e.g., the interval may be 30 degrees, 36 degrees, 40 degrees, 45 degrees, 60 degrees, 72 degrees, 90 degrees, 120 degrees, or 180 degrees. For instance, in an embodiment shown in FIG. 5A and FIG. 5B, the holes 23 are arranged at intervals of 180 degrees in a radial direction of the outer pipe 20. That is to say, the holes 23 are disposed on the opposite sides of the wall of the outer pipe 20, and FIG. 5A and FIG. 5B respectively illustrate the opposite sides. In another embodiment shown in FIG. 5C and FIG. 5D, the holes 23 are arranged at intervals of 90 degrees in a radial direction of the outer pipe 20, i.e., the holes 23 are symmetrically arranged in four directions of the wall of the outer pipe 20 at intervals of 90 degrees. In some embodiments, the holes 23 can also be asymmetrically arranged, and the aforementioned asymmetry means that the mirror image based on the axis of the outer pipe 20 is not in the same position, as shown in FIG. 5A and FIG. 5B. In the present disclosure, the distance between the holes 23 may be from 0.1 mm to 5 mm, such as 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm or 5 mm; and the dimension of the holes 23 can also be from 0.1 mm to 5 mm, such as 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, or 5 mm.

In at least one embodiment, the culture chamber 1 includes the inner pipe 30 and the outer pipe 20 having the holes 23 on the wall, and the flow pattern of the culture medium in the culture chamber 1 is shown in FIG. 6. The external culture medium enters into the outer pipe 20 from the inlet 11 and the third opening 21 of the outer pipe 20, and the external culture medium slides along the wall of the outer pipe 20 and flows to the outside of the outer pipe 20 through the holes 23 on the wall. The culture medium at a bottom 42 of the accommodating space enters the inner pipe 30 through the second opening 32 of the inner pipe 30 and leaves the accommodating space through the first opening 31 of the inner pipe 30 and the outlet 12, and is transported to a next culture chamber or the liquid storage chamber 2 by the pipelines 4.

FIG. 7 shows the bioreactor apparatus according to another embodiment of the present disclosure that connects a plurality of culture chambers 1a to if in series, in parallel, and a combination thereof, wherein the pump 3 serves as a power source for transporting the culture medium from the storage chamber 2 to the plurality of culture chambers 1a to if via the pipelines 4. The pipelines 4 are branched into two channels (or more channels in other embodiments) before being connected to the pump 3, and each channel can be individually pressurized by the pump 3. Then, the culture medium is transported to the culture chamber if through the upper channel of the pipelines 4. On the other hand, the culture medium is also transported to the culture chamber 1e through the lower channel of pipelines 4, then transported from the culture chamber 1e to the culture chamber 1d and by analogy till to the chamber 1a. After passing the plurality of culture chambers, the upper channel and lower channel of the pipelines 4 are converged and connected to the liquid storage chamber 2, so that the culture medium can be transported from the culture chambers back to the liquid storage chamber 2, forming a closed-loop system.

In at least one embodiment, as shown in FIG. 7, the pipelines 4 can be provided with a connector 5 for connecting pipelines 4 with each other or with a component such as the culture chambers 1, the liquid storage chamber 2, or the pump 3, etc. In some embodiments, the connector 5 may be a multi-way pipe and have an opening 50 that can be opened and closed for adding additional substances. For example, as shown in FIG. 7, the connector 5 is a three-way pipe for connecting the lower channel of the pipeline 4 with the culture chamber 1e, and one end of the three-way pipe is an opening 50 with a cap stopper 51 that can be unplugged to add additional substances (e.g., but not limited to, the culture medium, drugs, toxicants, samples, cytokines, growth factors, and so forth) into the culture medium. There is no restriction on where the connector 5 should be positioned; however, in some embodiments, it is preferably positioned close to the culture chambers for better observation of the effect of the additional substances on the cells cultured in the culture chambers.

The present disclosure also provides a bioreactor system that includes a bioreactor apparatus, a culture medium, and cells. Specifically, the aforementioned culture medium is added to the bioreactor apparatus, and the cells are cultured in each of the culture chambers. The culture can be conducted by suspension culture or adhesion culture. Suspension culture means that the cells are suspended in the culture medium without contacting and attaching to the surface of the wall or the surface of other components when cultured, whereas adhesion culture means that the cells are attached to the surface of the wall or the surface of other components when cultured. In at least one embodiment of the present disclosure, each of the culture chambers can culture the same or different types of cells.

In at least one embodiment of the present disclosure, as shown in FIG. 6 and FIG. 7, the inside of the culture chamber 1 is not fully filled with the culture medium, and thus the culture chamber 1 includes a gas section and a liquid section. The liquid in the liquid section is the culture medium that can be used to culture the cells; the gas section is located at the top part 41 of the culture chamber 1 that can provide space for the culture medium to slide (provided that the outer pipe 20 is disposed therein) or drip (provided that the outer pipe 20 is not disposed therein) from the inlet 11 of the culture chamber 1 to the liquid section.

In at least one embodiment of the present disclosure, the liquid section includes a scaffold. The scaffold is adapted to the cells and can be used for cell growth or adhesion to simulate a physiological microenvironment and provide a suitable environment for cell culture, growth, differentiation, etc. In some embodiments, the scaffold includes three-dimensional porous calcium alginate crosslinked bioscaffolds, three-dimensional porous collagen bioscaffolds, three-dimensional porous gelatin bioscaffolds, three-dimensional magnetic porous bioscaffolds, three-dimensional alginate/gelatin combined cell carriers, three-dimensional magnetic cell carriers, and other three-dimensional biological protein/polymer bioscaffolds.

In at least one embodiment of the present disclosure, the bioreactor system of the present disclosure can also include a sensor for sensing the ingredients of the culture medium. By culturing the cells via the bioreactor apparatus, the ingredients of the culture medium can be observed/analyzed to further study the state and behavior of the cells. For example, a specific substance (e.g., but not limited to, the culture medium, drugs, toxicants, samples, cytokines, growth factors, and so forth) that was added into the culture medium via the connector 5 shown in FIG. 7 may affect the cells and lead to changes in the state and behavior of the cells, and these changes can be observed by the sensor sensing the ingredients of the culture medium (including cell secretions). The ingredients to be sensed may be, e.g., proteins (including growth factors, paracrine factors, antibodies, or other cell-derived water soluble proteins), exosomes, glucose, hydrogen ion, oxygen, or nitrogenous wastes.

### Example 1

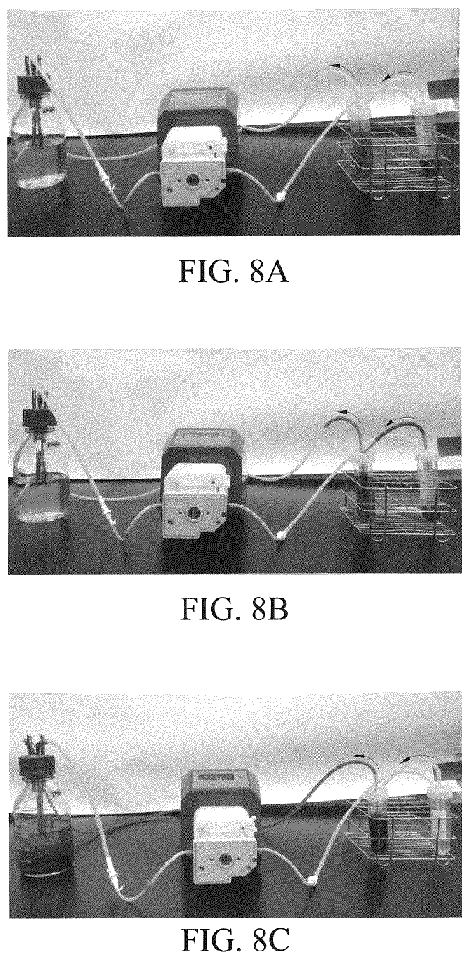

FIG. 8A to FIG. 8C are actual images of the bioreactor apparatus of example 1. As shown in FIG. 8A, the bioreactor apparatus includes a liquid storage chamber, pipelines, a pump, and two culture chambers. The liquid was added into the bioreactor apparatus and can be transported through the pipelines, wherein the liquid in the liquid storage chamber was transparent; the liquid in the first culture chamber (on the right side) was blue; the liquid in the second culture chamber (on the left side) was red. As shown in FIG. 8B, after the pump was started, the liquid in the liquid storage chamber was transported to the first culture chamber. At the same time, the blue liquid in the first culture chamber flowed out from the outlet and was transported to the second culture chamber, and the liquid in the second culture chamber also flowed out from the outlet and was transported back to the liquid storage chamber. At this time, it could be observed that the blue liquid in the first culture chamber was diluted and became lighter, and the red liquid in the second culture chamber was dyed dark by the blue liquid. As shown in FIG. 8C, more liquid was transported after a certain period of time; accordingly, the liquid color in the first culture chamber was much lighter, and the liquid color in the second culture chamber was much darker. In addition, the liquid in the liquid storage chamber also was dyed due to receiving the liquid from the second culture chamber. Afterward, through the continuous transportation of the liquid, the liquid color in the entire bioreactor apparatus became identical. The result shows that the bioreactor apparatus is a closed-loop system. That is to say, the liquid in the front culture chamber can be transported into the rear culture chamber, and the liquid in the rearmost culture chamber is transported back to the liquid storage chamber and again transported into the front culture chamber to form a circulatory system. The ingredients, e.g., the red and blue dyes, in each of the culture chambers will eventually be mixed and dispersed in the liquid within the entire apparatus.

### Example 2

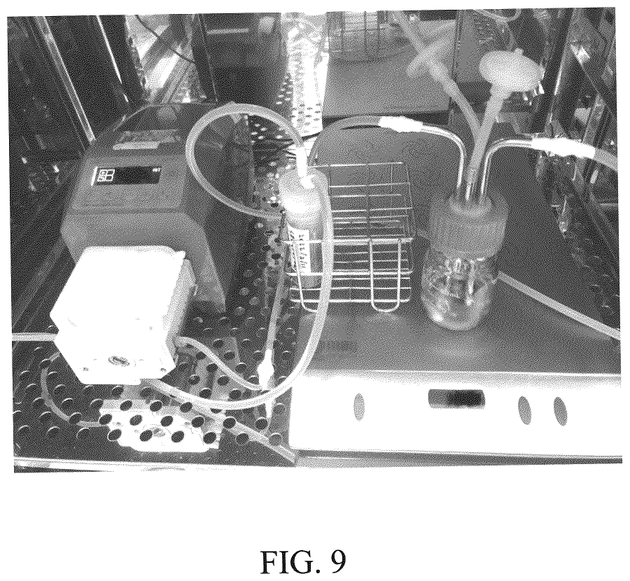

As shown in FIG. 9, the bioreactor apparatus included a liquid storage chamber, pipelines, a pump, and one culture chamber. The liquid was added into the bioreactor apparatus and can be transported through the pipelines. The inside of the culture chamber was not totally filled, and thus the culture chamber included a gas section and a liquid section. The gas section was located on the top part of the culture chamber, and the liquid section included a scaffold and a culture medium. The scaffold was adapted to cells, and the liquid was the culture medium that can be used for culturing the cells. After the pump was started, the culture medium in the liquid storage chamber was transported to the culture chamber, and the gas section can provide a space for the culture medium to slide (provided that the outer pipe is disposed therein) from an inlet of the culture chamber to the liquid section. At the same time, the culture medium in the culture chamber flowed out from an outlet, and was transported back to the liquid storage chamber to form a closed-loop system that provided a dynamic culture medium for cell growth and differentiation for an extended period.

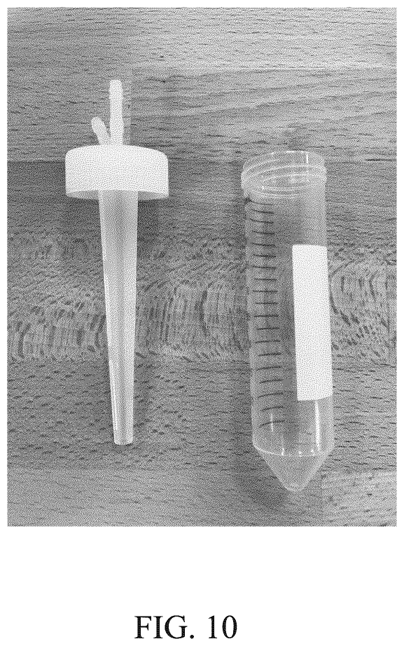

FIG. 10 is an actual image of the culture chamber in Examples 1 and 2. The culture chamber has a lid and a tube. The lid has an inlet and an outlet, and the lid has an inner pipe connected to the outlet and an outer pipe sleeved around the periphery of the inner pipe.

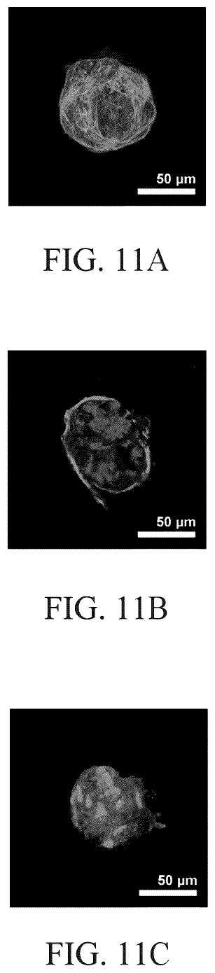

Mesenchymal stem cells cultured in the culture chamber of the bioreactor apparatus were collected and the fluorescence microscopy image thereof was taken. As shown in FIG. 11A, wherein green fluorescence showed F-actins labeled by Phalloidin, and blue fluorescence showed cell nuclei stained by Hoechst 33342. As shown in FIG. 11B, green fluorescence showed F-actins labeled by Phalloidin; red fluorescence showed healthy mesenchymal stem cells labeled by 5,5,6,6-Tetrachloro-1,1,3,3-tetraethylbenzimidazolylcarbocyanine iodide (a JC-1 mitochondria dye); blue fluorescence showed cell nuclei stained by Hoechst 33342. As shown in FIG. 11C, green fluorescence was live cells labeled by Calcein AM, and red fluorescence showed dead cells labeled by Propidium Iodide (PI). The results show that the mesenchymal stem cells were aggregated into cell spheroids in suspension culture in the apparatus of the present disclosure and exhibited excellent cell viability.

### Example 3

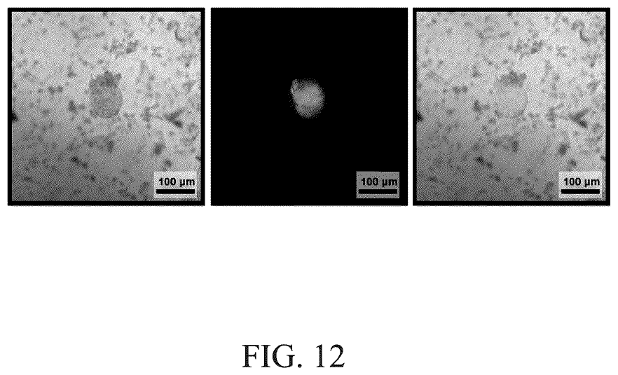

The same disposal as the aforementioned Example 2 was adapted, except that the cochlear progenitor cells were cultured in the culture chamber. The cells culture in the culture chamber were collected, and fluorescence microscopy image thereof was taken. As shown in FIG. 12, the green fluorescence was green fluorescent protein (GFP). The result shows that the cochlear progenitor cells aggregated into cell spheroids similar to those in Example 2 after being cultured in suspension with the apparatus of the present disclosure, exhibiting excellent cell viability.

What is claimed is:

1. A bioreactor apparatus, comprising:
a liquid storage chamber for storing a culture medium;
a pump;
a culture chamber having an accommodating space to accommodate the culture medium and a cell to be cultured; and
a pipeline connected to the liquid storage chamber, the pump, and the culture chamber to form a closed loop,
wherein the pump is configured to provide pressure to drive the flow of the culture medium in the closed loop.

2. The bioreactor apparatus of claim 1, comprises a plurality of the culture chambers connected to each other in series, in parallel, or a combination thereof.

3. The bioreactor apparatus of claim 1, wherein the culture chamber has an opposite top part and bottom part, the top part has an inlet allowing the culture medium to enter the accommodating space of the culture chambers, and the top part has an outlet allowing the culture medium at the bottom part to leave the accommodating space of the culture chambers.

4. The bioreactor apparatus of claim 3, wherein the culture chamber comprises a lid and a tube, the lid is disposed on the top part and forms the inlet and the outlet.

5. The bioreactor apparatus of claim 4, wherein the inlet is disposed obliquely relative to the lid, and the outlet is perpendicular to the lid.

6. The bioreactor apparatus of claim 5, wherein the lid and the inlet form an angle of 30 degrees to 60 degrees.

7. The bioreactor apparatus of claim 3, wherein the culture chamber further comprises an inner pipe having an opposite first opening and a second opening, the first opening is connected to the outlet, and the second opening is located at the bottom part of the culture chamber.

8. The bioreactor apparatus of claim 7, wherein the inner pipe is configured for allowing the culture medium in the accommodating space to enter the inner pipe through the second opening and to leave the accommodating space through the first opening and the outlet.

9. The bioreactor apparatus of claim 7, wherein the culture chamber further comprises an outer pipe sleeved around the inner pipe, the outer pipe has an opposite third opening and fourth opening, and the third opening is connected to the inlet.

10. The bioreactor apparatus of claim 9, wherein the outer pipe is configured for allowing the culture medium to enter the outer pipe through the inlet and the third opening.

11. The bioreactor apparatus of claim 9, wherein a diameter of the third opening diameter is larger than a diameter of the fourth opening.

12. The bioreactor apparatus of claim 9, wherein the outer pipe has a plurality of holes positioned on a wall of the outer pipe to allow the culture medium to flow out of the outer pipe through the holes.

13. The bioreactor apparatus of claim 12, wherein the holes are arranged at intervals of 30 degrees to 180 degrees in a radial direction of the outer pipe.

14. The bioreactor apparatus of claim 12, wherein the holes are asymmetrically arranged.

15. The bioreactor apparatus of claim 1, further comprising a connector for connecting the pipeline, wherein the connector has an openable and closable opening for adding an additional substance into the pipeline.

16. A bioreactor system, being a closed-loop system and comprising:
the bioreactor apparatus of claim 1;
a culture medium stored in the liquid storage chamber to be transported through the pipelines to the culture chambers; and
a cell cultured in the culture chambers.

17. The bioreactor system of claim 16, wherein the culture chamber comprises a liquid section and a gas section, and the liquid section comprises the culture medium for culturing the cells.

18. The bioreactor system of claim 16, further comprises a sensor for sensing an ingredient of the culture medium.

19. A method for culturing a cell, comprising:
providing the bioreactor apparatus of claim 1; and
culturing the cell in the culture chamber.

20. The method of claim 19, further comprising:
loading the culture medium in the liquid storage chamber and the culture chamber; and
starting the pump to provide a constant pressure, a periodic pressure, or a pulsatory pressure to drive the flow of the culture medium in the closed loop.
